(19) United States
(12) Patent Application Publication
(10) Pub. No.: US 2022/0133650 A1
(43) Pub. Date: May 5, 2022

(54) COMBINATION THERAPY FOR TREATMENT OF BRAIN DISORDERS

(71) Applicant: B. G. NEGEV TECHNOLOGIES AND APPLICATIONS LTD., AT BEN-GURION UNIVERSITY, Beer-sheva (IL)

(72) Inventors: Alon FRIEDMAN, Scotch Village (CA); Ofer PRAGER, Beer-Sheva (IL); Yehuda VAZANA, Rehovot (IL)

(21) Appl. No.: 17/428,976

(22) PCT Filed: Feb. 6, 2020
(86) PCT No.: PCT/IL2020/050149

§ 371 Date: Aug. 6, 2021

Related U.S. Application Data
(60) Provisional application No. 62/801,727, filed on Feb. 6, 2019.

(51) Int. Cl. A61K 31/13; A61K 31/4178; A61K 45/06; A61P 25/28; A61P 25/08

(52) U.S. Cl. CPC A61K 31/13; A61K 31/4178; A61P 25/08; A61P 25/28; A61K 45/06

(57) ABSTRACT

A combination therapy, comprising a pharmaceutical composition of an N-Methyl D-Aspartate receptor (NMDA) receptor blocker and a Transforming Growth Factor beta (TGF-β) receptor antagonist, for treatment of brain diseases associated with BBB dysfunction.

### CROSS-REFERENCE TO RELATED APPLICATIONS

This application claims the benefit of priority of U.S. Provisional Patent Application No. 62/801,727 filed Feb. 6, 2019, the contents of which are incorporated herein by reference in their entirety.

### FIELD OF INVENTION

The present invention is in the field of treatment of brain disorders.

### BACKGROUND OF THE INVENTION

The blood-brain barrier (BBB) is a highly specific interface (or complex mechanism) that separates the circulating blood from the extracellular fluid in the brain. The BBB allows a passive diffusion of lipophilic molecules as well as a selective transport of molecules (e.g., nutrients, etc.) across it. The selective nature of the BBB allows the formation of a unique extracellular milieu within brain neuropil, essential for normal brain function.

In most common brain disorders, including epilepsy, traumatic brain injury, stroke, and neurodegenerative diseases, the BBB function may be impaired initiating a neural network reorganization, neural dysfunction and degeneration. Despite the clear need for a treatment for these severe physiological conditions, to the best of our knowledge, there is no reported medication for BBB-dysfunction.

### SUMMARY OF THE INVENTION

The present invention is directed to a combination therapy for reducing the permeability of the blood-brain-barrier (BBB), in a subject in need thereof. In some embodiments, the invention is directed to a composition comprising an N-Methyl D-Aspartate receptor (NMDA) receptor blocker and a Transforming Growth Factor beta (TGF-β) receptor antagonist.

In one aspect, there is provided a pharmaceutical composition comprising a therapeutically effective amount of an NMDA receptor blocker and a therapeutically effective amount of a TGF-β receptor antagonist.

In one embodiment, the NMDA receptor blocker is selected from the group consisting of: Memantine, D-2-amino-5-phosphonopentanoate (AP5), 2-amino-7-phosphonoheptanoic acid (AP7), 3-[(R)-2-carboxypiperazin-4-yl]-prop-2-enyl-1phosphonic acid, (2S, 4R)-4-(phosphonomethyl)piperidine-2-carboxylic acid, Amantadine, Nitromemantine, and Symmetrel or any combination thereof.

In one embodiment, the TGF-β receptor antagonist is selected from the group consisting of: Losartan, 4-(5-Benzol[1,3]dioxol-5-yl-4-pyridin-2-yl-1H-imidazol-2-yl)-benzamide, 2-(3-(6-Methylpyridin-2-yl)-1H-pyrazol-4-yl)-1,5-naphthyridine, Candesartan, and Telmisartan or any combination thereof.

In one embodiment, the NMDA receptor blocker and the TGF-β receptor antagonist are present in the pharmaceutical composition at a ratio ranging from 1:0.1 to 1:15.

In one embodiment, the pharmaceutical composition further comprises a pharmaceutically acceptable carrier.

In one embodiment, the pharmaceutical composition is intended for use in the prevention or treatment of a disorder associated with blood-brain-barrier (BBB) dysfunction.

In one embodiment, the disorder associated with BBB dysfunction is selected from the group consisting of seizures, status epilepticus, BBB pathology, traumatic brain injury, neurodegenerative diseases and brain ischemia or a combination thereof.

In another aspect, there is provided a combination of the NMDA receptor blocker and the TGF-β receptor antagonist for use in the prevention or treatment of a disorder associated with BBB dysfunction.

In one embodiment, the NMDA receptor blocker is formulated within a first pharmaceutical composition and the TGF-β receptor antagonist is formulated within a second pharmaceutical composition.

In another aspect, there is provided a method for reducing the BBB permeability in a subject in need thereof, comprising contacting the subject with an effective amount of an NMDA receptor blocker and an effective amount of a TGF-β receptor antagonist thereby reducing the BBB permeability in the subject.

In one embodiment, the NMDA receptor blocker and the TGF-β receptor antagonist are administered at a ratio ranging from 1:0.1 to 1:15.

In another aspect, there is provided a method for increasing or prolonging the therapeutic efficacy of the NMDA receptor blocker in a subject in need thereof, comprising administering to said subject a pharmaceutical composition comprising the TGF-β receptor antagonist.

In one embodiment, the NMDA receptor blocker is administered at a dosage of 0.1-40 mg/kg.

In one embodiment, the TGF-β receptor antagonist is administered at a dosage of 0.1-60 mg/kg.

In one embodiment, the NMDA receptor blocker is selected from the group consisting of: Memantine, D-2-amino-5-phosphonopentanoate (AP5), 2-amino-7-phosphonoheptanoic acid (AP7), 3-[(R)-2-carboxypiperazin-4-yl]-prop-2-enyl-1phosphonic acid, (2S,4R)-4-(phosphonomethyl) piperidine-2-carboxylic acid, Amantadine, Nitromemantine, and Symmetrel or any combination thereof.

In one embodiment, the TGF-β receptor antagonist is selected from the group consisting of: Losartan, 4-(5-Benzol[1,3]dioxol-5-yl-4-pyridin-2-yl-1H-imidazol-2-yl)-benzamide, 2-(3-(6-Methylpyridin-2-yl)-1H-pyrazol-4-yl)-1,5-naphthyridine, Candesartan, and Telmisartan or any combination thereof.

In one embodiment, the subject is afflicted with BBB dysfunction.

In one embodiment, BBB dysfunction is selected from the group consisting of epilepsy, traumatic brain injury, neurodegenerative diseases and brain ischemia.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-G: Quantitative analysis of electrocorticography and fluorescent imaging for detection of BBB dysfunction during repeated seizure activity. FIG. 1A represents electrocorticography (ECoG) recorded from the exposed rat cortex. The addition of 4AP to the ACSF perfusing the cortex generates a state of repeated seizures (status epilepticus, SE, indicated by dashed arrows). FIG. 1B represents spectral analysis of 1 min ECoG during baseline (indicated by red circles), at 10 min (indicated by black rectangles) and 30 min (indicated by blue triangles) from seizure onset (SO). FIG. 1C represents difference from pre-SO in Mann-Whitney rank at 0-10 min from seizure onset (grey bars on left for each parameter) and 10-30 min from seizure onset (black bars on right for each parameter), calculated for mean spectral power (MSP), dominant frequency (DF) and energy of ECoG. FIG. 1D represents fluorescent angiography of the exposed rat cortex following i.v. administration of sodium fluorescein. FIG. 1E represents image segmentation differentiates between vascular and extra-vascular compartments (indicated by blue labelling). Additionally, a region of interest (ROI) in a primary vessel is manually selected (indicated by red, dashed frame). FIG. 1F represents IT curves of the primary vessel (indicated by triangles) and extra-vascular compartment (indicated by rectangles), calculated at baseline (top) and 30′ from seizure onset (bottom). Black, dashed arrow indicates time phase of the curve, from which permeability index (PI) is extrapolated. FIG. 1G represents detection of extra-vascular pixels exhibiting PI>1 (red) at baseline (left) and 30 min from seizure onset (right). * p<0.05, **p<0.01.

FIGS. 2A-C: Combined memantine and Los treatment achieves protection against both fast and slow BBB-dysfunction in acute-phase SE. FIG. 2A represents indication of extra-vascular pixels exhibiting PI>1 (red) at baseline (left column), 10 min (middle column) and 30 min (right column) from seizure onset. The analysis was done for the untreated group (top row), NMDAr-A stand-alone treated group (middle row) and memantine+Losartan (Los) treated group (bottom row). FIG. 2B represents PI shift from baseline at 10 and 30 min from seizure onset, calculated for the untreated group (blue square), Los stand-alone treated group (black square), NMDAr-A stand-alone treated group (red circle) and memantine+Los treated group (green triangle). FIG. 2C represents ECoG Mann-Whitney rank analysis (see Materials and Methods) for the untreated (grey bars on left for each parameter) and memantine+Los treated (black bars on right for each parameter) groups. Differences from pre-seizure onset in Mann-Whitney rank, at 10-30 min from seizure onset, calculated for mean spectral power (MSP), dominant frequency (DF) and energy of ECoG. * p<0.05, ** p<0.01.

FIGS. 3A-B: Combined antagonism of NMDA and TGF-β receptors achieves protection against both fast and slow BBB dysfunction in acute-phase SE. FIG. 3A represents Mean±SEM PI shift from pre-substance application (baseline), calculated for application of angiotensin II (ATII, circle) and transforming growth factor beta 1 (TGF-β1, triangle) FIG. 3B represents mean±SEM PI shift from baseline at 10 and 30 min from seizure onset, calculated for application of 4AP (indicated by squares) and for addition of AP5 and SJN (indicated by circles). SE was induced by 4AP addition to the ACSF perfusing the cortex. The addition of AP5 and SJN to the ACSF prevents seizure-induced PI increase. * p<0.05, ** p<0.01.

FIGS. 4A-D: Contrast enhanced T1-weighted MRI following thrombotic stroke and offline analysis for detection of BBB detection. FIG. 4A represents T1-weighted MRI of the rat head, 48 h following photo-induced thrombotic stroke, prior to ABLAVAR administration and FIG. 4B represents 30 min following ABLAVAR administration. FIG. 4C represents manual selection of a reference region (green, indicated by an arrow) in the temporal muscle. FIG. 4D represents detection of brain pixels with abnormally high-level permeability (BBB dysfunction (BBBD)—blue colour, indicated by an arrow) in comparison to reference.

FIGS. 5A-B: Combined memantine+Los treatment reduces sub-acute BBB dysfunction within the peri-ischemic cortex. FIG. 5A represents T1-weighted imaging of the anesthetized rat head, 48 h following photo-induced thrombotic stroke, overlaid with detection of abnormally high-level vascular permeability (BBB dysfunction) in the brain (blue colour coded voxels, areas indicated by arrows). Analysis was done for animals treated (i.p via osmotic pumps) with 0.9% NaCl (vehicle, upper left), memantine stand-alone (memantine, upper right), losartan stand-alone (Los, lower left) and memantine and losartan combination (memantine+Los, lower right). FIG. 5B represents mean+SEM relative BBBD volume (100* # BBBD voxels/# brain voxels) for all treated groups. *p<0.05.

FIGS. 6A-D: T2-weighted MRI following thrombotic stroke and offline analysis for lesion detection. FIG. 6A represents T2-weighted MRI of the rat head, 48 h following photo-induced thrombotic stroke. FIG. 6B represents image segmentation excluding brain region (blue, indicated by an arrow). FIG. 6C represents manual selection of a control region (green, indicated by an arrow). FIG. 6D represents detection of over-enhanced brain pixels (red, indicated by an arrow) in comparison to control.

FIGS. 7A-B: Combined memantine+Los treatment reduces sub-acute oedema in the peri-ischemic cortex. FIG. 7A represents T2-weighted imaging of the anesthetized rat head, 48 h following photo-induced thrombotic stroke, overlaid with lesion detection (red colour coded voxels areas indicated by arrows), treated (i.p via osmotoic pumps) with 0.9% NaCl (n=5) (vehicle, upper left), memantine stand-alone 40 mg/kg (n=8) (memantine, upper right), losartan stand-alone 60 mg/kg (n=5) (losartan, lower left), and memantine and losartan combination (n=6) (memantine+Los, lower right). FIG. 7B represents mean+SEM relative lesion volume (100*# lesion voxels/# brain voxels) for all treated groups, *p<0.05.

### DETAILED DESCRIPTION OF THE INVENTION

The present invention is directed to a kit or a composition comprising an NMDA receptor blocker and a TGF-β receptor antagonist and use thereof, such as for modulating the permeability of the blood-brain-barrier (BBB), in a subject in need thereof. The invention is further directed to a combination therapy of an NMDA receptor blocker and a TGF-β receptor antagonist such as for modulating the permeability of the BBB, in a subject in need thereof.

The present invention is also directed to a method for increasing or enhancing the therapeutic efficacy of an NMDA receptor blocker administered to a subject in need thereof, comprising administering to the subject a pharmaceutical composition comprising a TGF-β receptor antagonist.

The present invention is based, in part, on the finding that a combination of a TGF-β receptor antagonist and an NMDA receptor blocker reduced the permeability of BBB, in vivo. The invention is further based, in part, on the finding that the synergy of a TGF-β receptor antagonist and an NMDA receptor blocker is beneficial in preventing micro- and macro-molecular BBB leakage after brain ischemia, and additionally ameliorates both short-, and long-term seizure-induced BBB dysfunction.

Thus, the present invention provides a combination therapy of an NMDA receptor blocker and a TGF-β receptor antagonist for prevention or treatment of a disorder associated with BBB dysfunction in a subject in need thereof.

### NMDA Receptor Blocker

In some embodiments, the present invention is directed to a combined therapy comprising a pharmaceutical composition comprising an NMDA receptor blocker, and methods of use thereof.

As used herein, the term “NMDA receptor blocker” encompasses any compound that deactivates the N-Methyl D-Aspartate (NMDA) receptor activity. In some embodiments, an NMDA receptor antagonist of the present invention is a molecule active in reducing BBB permeability.

In some embodiments, an NMDA receptor blocker is an NMDA receptor antagonist. In some embodiments, an NMDA receptor antagonist is selected from a competitive antagonist, or an uncompetitive antagonist, an allosteric antagonist or a glycine antagonist. In some embodiments, the NMDA receptor is a glutamate-activated NMDA receptor.

Non-limiting examples of NMDA receptor blockers include, but are not limited to: D-2-amino-5-phosphonopentanoate (AP5), 2-amino-7-phosphonoheptanoic acid (AP7), 3-[(R)-2-carboxypiperazin-4-yl]-prop-2-enyl-1phosphonic acid (CPPene), (2S, 4R)-4-(phosphonomethyl)piperidine-2-carboxylic acid (Selfotel), Memantine, Amantadine, Nitromemantine, and Symmetrel including any derivative, isomer or a combination thereof.

Additional NMDA receptor blockers are known in the art and disclosed in US Application No. 20180214462.

As used herein, the term derivative is related to a chemical derivative of the active agent (e.g., memantine and/or losartan) having a biological or a therapeutic activity similar to the activity of the active agent. In some embodiments, derivative is a pharmaceutically active derivative. In some embodiments, a derivative of the NMDA receptor blocker is a molecule structurally related to an NMDA receptor blocker and being capable of reducing NMDA receptor activity. In some embodiments, a derivative of the TGF-β receptor antagonist is a molecule structurally related to an TGF-β receptor antagonist and being capable of reducing TGF-β receptor activity.

In some embodiments, any of the NMDA receptor blocker and the TGF-β receptor antagonist are in a from of a pharmaceutically acceptable salt. In some embodiments, pharmaceutically acceptable salt comprises any of the NMDA receptor blocker and the TGF-β receptor antagonist and a pharmaceutically acceptable anion.

Non-limiting examples of pharmaceutically acceptable anions include but are not limited to: acetate, aspartate, benzenesulfonate, benzoate, bicarbonate, carbonate, halide (such as bromide, chloride, iodide, fluoride), bitartrate, citrate, salicylate, stearate, succinate, sulfate, tartrate, decanoate, edetate, fumarate, gluconate, and lactate or any combination thereof.

In some embodiments, the NMDA receptor blocker is administered at a dosage of 0.1-60 mg/kg. In some embodiments, the NMDA receptor blocker is administered at a dosage of 0.1-4 mg/kg. In some embodiments, the NMDA receptor blocker is administered at a dosage of 0.1-0.5 mg/kg. In some embodiments, the NMDA receptor blocker is administered at a dosage of 0.4-1 mg/kg. In some embodiments, the NMDA receptor blocker is administered at a dosage of 0.8-2 mg/kg. In some embodiments, the NMDA receptor blocker is administered at a dosage of 1-3 mg/kg. In some embodiments, the NMDA receptor blocker is administered at a dosage of 2-4 mg/kg. In some embodiments, the NMDA receptor blocker is administered at a dosage of 5-10 mg/kg. In some embodiments, the NMDA receptor blocker is administered at a dosage of 6-7 mg/kg. In some embodiments, the NMDA receptor blocker is administered at a dosage of 10-20 mg/kg. In some embodiments, the NMDA receptor blocker is administered at a dosage of 20-30 mg/kg. In some embodiments, the NMDA receptor blocker is administered at a dosage of 30-40 mg/kg. In some embodiments, the dosage comprises daily dosage.

In some embodiments, memantine is administered at a daily dosage of 1-20 mg/kg. In some embodiments, memantine is administered at a daily dosage of 1-10 mg/kg. In some embodiments, memantine is administered at a daily dosage of 5-10 mg/kg. In some embodiments, memantine is administered at a daily dosage of 5-8 mg/kg. In some embodiments, memantine is administered at a daily dosage of 6-7 mg/kg. In some embodiments, the terms “dose” or “dosage” are as described hereinbelow.

### TGF-β Receptor Antagonist

In some embodiments, the present invention is directed to a combined therapy comprising a pharmaceutical composition comprising a TGF-β receptor antagonist, and methods of use thereof.

As used herein, the term “TGF-β receptor antagonist” encompasses a compound that binds to a Transforming Growth Factor beta (TGF-β) receptor and inhibits a TGF-β mediated signaling activity. In some embodiments, a TGF-β receptor antagonist exhibits an additional activity as an Angiotensin II type 1 receptor (AT1) antagonist. In some embodiments, a TGF-β receptor antagonist is a molecule active in reducing BBB permeability.

In some embodiments a TGF-β receptor antagonist is selected from but not limited to a group containing ALK-1 inhibitors and ALK-5 inhibitors. Non-limiting examples of TGF-β receptor antagonists include but are not limited to: Losartan, Candesartan, Telmisartan, 4-(5-Benzol[1,3]dioxol-5-yl-4-pyridin-2-yl-1H-imidazol-2-yl)-benzamide (SB431542), 2-(3-(6-Methylpyridin-2-yl)-1H-pyrazol-4-yl)-1,5-naphthyridine (SJN2511) including any derivative, isomer or a combination thereof.

In some embodiments, the TGF-β receptor antagonist is administered at a dosage of 0.1-60 mg/kg. In some embodiments, the TGF-β receptor antagonist is administered at a dosage of 0.1-40 mg/kg. In some embodiments, the TGF-β receptor antagonist is administered at a dosage of 0.1-0.5 mg/kg. In some embodiments, the TGF-β receptor antagonist is administered at a dosage of 0.4-1 mg/kg. In some embodiments, the TGF-β receptor antagonist is administered at a dosage of 0.8-2 mg/kg. In some embodiments, the TGF-β receptor antagonist is administered at a dosage of 1-5 mg/kg. In some embodiments, the TGF-β receptor antagonist is administered at a dosage of 1-10 mg/kg. In some embodiments, the TGF-β receptor antagonist is administered at a dosage of 10-20 mg/kg. In some embodiments, the TGF-β receptor antagonist is administered at a dosage of 8-12 mg/kg. In some embodiments, the TGF-β receptor antagonist is administered at a dosage of 20-30 mg/kg. In some embodiments, the TGF-β receptor antagonist is administered at a dosage of 30-40 mg/kg. In some embodiments, the TGF-β receptor antagonist is administered at a dosage of 40-50 mg/kg. In some embodiments, the TGF-β receptor antagonist is administered at a dosage of 50-60 mg/kg.

In some embodiments, losartan is administered at a dosage of 8-12 mg/kg. In some embodiments, losartan is administered at a dosage of 1-20 mg/kg. In some embodiments, losartan is administered at a dosage of 10-30 mg/kg. In some embodiments, the terms “dose” or “dosage” are as described hereinbelow.

In some embodiments, the compounds of the invention include any polymorph thereof. In some embodiments, the polymorph is a therapeutically or biologically active polymorh.

In some embodiments, the compounds described herein are chiral compounds (i.e. possess an asymmetric carbon atom). In some embodiments, isomer comprises a diastereomer, a geometric isomer and an individual isomer. As used herein, the term “isomer” encompasses any therapeutically or biologically active isomer.

In some embodiments, a chiral compound described herein is in form of a racemic mixture. In some embodiments, a chiral compound is in form of a single enantiomer, with an asymmetric carbon atom having the R configuration. In some embodiments, a chiral compound is in form of a single enantiomer, with an asymmetric carbon atom having the S configuration as described hereinabove.

In some embodiments, a chiral compound is in form of a single enantiomer with enantiomeric purity of more than 70%. In some embodiments, a chiral compound is in form of a single enantiomer with enantiomeric purity of more than 80%. In some embodiments, a chiral compound is in form of a single enantiomer with enantiomeric purity of more than 90%. In some embodiments, a chiral compound is in form of a single enantiomer with enantiomeric purity of more than 95%.

In some embodiments, the compounds described herein can exist in unsolvated form as well as in solvated form, including hydrated form. In general, the solvated form is equivalent to the unsolvated form and is encompassed within the scope of the present invention. Certain compounds of the present invention may exist in multiple crystalline or amorphous forms. In general, all physical forms are equivalent for the uses contemplated by the present invention and are intended to be within the scope of the present invention.

The term “solvate” refers to a complex of variable stoichiometry (e.g., di-, tri-, tetra-, penta-, hexa-, and so on), which is formed by a solute (the conjugate described herein) and a solvent, whereby the solvent does not interfere with the biological activity of the solute. Suitable solvents include, for example, ethanol, acetic acid and the like.

The term “hydrate” refers to a solvate, as defined hereinabove, where the solvent is water.

### Methods of Use

In some embodiments, the present invention is directed to a method for inducing blood-brain-barrier (BBB) protection in a subject in need thereof. In some embodiments, provided herein is a method for reducing or inhibiting the BBB permeability in a subject in need thereof. In some embodiments, provided herein is a method for reducing or inhibiting the BBB dysfunction in a subject in need thereof. In some embodiments, provided herein is a method for preventing a BBB pathology associated with a BBB dysfunction in a subject in need thereof. In some embodiments, provided herein a method for preventing or treating a BBB pathology, in a subject in need thereof. In some embodiments, provided herein a method for preventing or treating an increased brain vascular permeability. In some embodiments, provided herein a method for preventing or treating brain vascular leakage.

As used herein, the term “BBB protection” refers to a method of reducing the BBB permeability, such as for preventing blood constituents, normally restricted from the brain (e.g. serum proteins, small molecules or ions, circulating cells), to cross the BBB and accumulate within the brain. An increased BBB permeability may be associated with BBB dysfunction.

As used herein, the term “BBB dysfunction” refers to structural changes (e.g. within a basement membrane, a glial foot, cell-cell junctions, endothelial cells, pericytes, astrocytes, oligodendrocytes, astrocytes, ependymal cells, Schwann cells, microglia, satellite cells or other glial cells) or functional changes (e.g. influx transporter dysfunction, efflux transporter dysfunction, degeneration of BBB components, altered expression of proteins associated with junction formation). The impaired ability of brain blood vessels to separate the circulating blood from the extracellular fluid (as under healthy conditions) can contribute to severe neural damage.

In some embodiments, BBB dysfunction comprises increased brain vascular permeability. In some embodiments, BBB dysfunction comprises structural or functional changes resulting from a cerebral infarction, a brain ischemia or from a stroke. In some embodiments, BBB dysfunction comprises structural or functional changes resulting from an epileptic or non-epileptic seizure. In some embodiments, BBB dysfunction comprises structural or functional changes resulting from brain injury. In some embodiments, BBB dysfunction comprises structural or functional changes resulting from concussion.

In some embodiments, provided herein is a method for preventing, reducing or treating pathologies, including but not limited to: stroke, cognitive decline, Alzheimer's disease, non-Alzheimer's neurodegenerative diseases, acute liver failure, multiple sclerosis, meningitis, HIV, diabetes, a movement disorder, a depressive and/or psychotic disorder, cerebral malaria, Parkinson's disease, traumatic and surgical brain injury, concussion, brain oedema, peripheral nerve injury, brain cancer, epilepsy and chronic pain.

In some embodiments, provided herein is a method for extending the effect of BBB protection by an NMDA receptor blocker in a subject in need thereof, comprising administering to a subject a pharmaceutical composition comprising a TGF-β receptor antagonist.

In some embodiments, there is a method is for reducing early-phase BBB dysfunction comprising administering the NMDA receptor blocker to the subject in nedd thereof. In some embodiments, there is a method is for extending the effect of the NMDA receptor blocker comprising administering to the subject a TGF-β receptor antagonist.

In some embodiments, provided herein is a method for reducing or inhibiting acute-phase seizure-induced BBB dysfunction by administering to a subject a combination of a TGF-β receptor antagonist and an NMDA receptor blocker.

In some embodiments, provided here a method for preventing, reducing or inhibiting the early-phase BBB dysfunction and the delayed-phase BBB dysfunction. In some embodiments, administering to a subject a combination of a TGF-β receptor antagonist and an NMDA receptor blocker reduces or inhibits the early-phase BBB dysfunction and the delayed-phase BBB dysfunction.

As used herein, the term “early-phase BBB dysfunction” refers to damage induced BBB opening, governed by glutamatergic activation of NMDA-receptors. As used herein, the term “delayed-phase BBB permeability” refers to damage induced BBB opening governed by activation of a TGF-β receptor. In some embodiments, damage is seizure-induced damage. In some embodiments, damage is infarct-induced damage.

In some embodiments, provided herein is a method for enhancing the reduction or inhibition of the BBB permeability or dysfunction by an NMDA receptor blocker in a subject in need thereof, comprising administering to a subject a pharmaceutical composition comprising a TGF-β receptor antagonist. In some embodiments, the BBB permeability is decreased or inhibited for macro-molecules and/or micro-molecules. In some embodiments, administering to a subject a pharmaceutical composition comprising an NMDA receptor blocker reduces or inhibits the BBB permeability for micro-molecules. In some embodiments, administering to a subject a combination of a TGF-β receptor antagonist and an NMDA receptor blocker reduces or inhibits the BBB permeability for both macro-molecules and micro-molecules (e.g. small molecule).

As used herein, the term “small-molecule” refers to any molecule less than 5000 Dalton (D). Examples include, but are not limited to, chemicals, nutraceuticals, pharmaceuticals, dyes, tracers, vitamins, along with food diet supplements, and combinations thereof. As used herein, the term “macromolecule” refers to any molecule greater than 5000 D. Examples include, but are not limited to, large biological molecules, biopolymers, proteins and combinations thereof.

In some embodiments, BBB protection is irreversible for more than 72 hours since damage onset. In some embodiments, BBB protection is irreversible in the range from ten minutes to 72 hours since damage onset. In some embodiments, BBB protection is irreversible in the range from ten minutes to 48 hours since damage onset. In some embodiments, BBB protection is irreversible in the range from ten minutes to 24 hours since damage onset. In some embodiments, BBB protection is irreversible in the range from ten minutes to 14 hours since damage onset. In some embodiments, BBB protection is irreversible in the range from ten minutes to 5 hours since damage onset. In some embodiments, BBB protection is irreversible in the range from ten minutes to 30 minutes since damage onset.

In some embodiments, a subject is a human subject. In some embodiments, a subject is an animal. In some embodiments, a subject is a farm animal. In some embodiments, a subject is a pet.

In some embodiments, a subject in need of a method for reducing and/or inhibiting BBB permeability or dysfunction suffers from brain seizures. In some embodiments, a subject in need of a method for reducing and/or inhibiting BBB permeability or dysfunction suffers from epileptic or non-epileptic seizures. In some embodiments, a subject in need of a method for reducing and/or inhibiting BBB permeability or dysfunction suffers from reoccurring brain seizures (e.g., status epilepticus). In some embodiments, a subject in need of a method for reducing and/or inhibiting BBB permeability or dysfunction suffers from a brain trauma. In some embodiments, a subject in need of a method for reducing BBB permeability or dysfunction suffers from a brain oedema. In some embodiments, a subject in need of a method for reducing BBB permeability or dysfunction suffers from a cerebral oedema. In some embodiments, a subject in need of a method for reducing BBB permeability or dysfunction suffers from posterior reversible encephalopathy syndrome. In some embodiments, a subject in need of a method for reducing BBB permeability or dysfunction suffers from cognitive decline. In some embodiments, a subject in need of a method for reducing BBB permeability or dysfunction suffers from Parkinson's disease or other movement disorders. In some embodiments, a subject in need of a method for reducing BBB permeability or dysfunction suffers from depression.

In some embodiments, a subject in need of a method for reducing and/or inhibiting BBB permeability or dysfunction suffers from a brain injury. In some embodiments, a subject in need of a method for reducing and/or inhibiting BBB permeability or dysfunction suffers from stroke (e.g. brain ischemia, cerebral infarction). In some embodiments, a subject in need of a method as described herein is afflicted with a BBB pathology. In some embodiments, a subject in need of a method for reducing and/or inhibiting BBB permeability or dysfunction suffers from Meningitis and/or encephalitis. In some embodiments, a subject in need of a method for reducing BBB permeability or dysfunction suffers from bacterial meningitis. In some embodiments, a subject in need of a method for reducing BBB permeability or dysfunction suffers from viral meningitis. In some embodiments, a subject in need of a method for reducing and/or inhibiting BBB permeability or dysfunction suffers from Brain abscess. In some embodiments, a subject in need of a method for reducing and/or inhibiting BBB permeability or dysfunction suffers from epilepsy. In some embodiments, a subject in need of a method for reducing and/or inhibiting BBB permeability or dysfunction suffers from multiple sclerosis. In some embodiments, a subject in need of a method for reducing and/or inhibiting BBB permeability or dysfunction suffers from Neuromyelitis optica. In some embodiments, a subject in need of a method for reducing and/or inhibiting BBB permeability or dysfunction suffers from Progressive multifocal leukoencephalopathy (PML). In some embodiments, a subject in need of a method for reducing and/or inhibiting BBB permeability or dysfunction suffers from HIV-related brain disorder (e.g. encephalitis or cognitive decline). In some embodiments, a subject in need of a method for reducing and/or inhibiting BBB permeability or dysfunction suffers from cerebral malaria. In some embodiments, a subject in need of a method for reducing and/or inhibiting BBB permeability or dysfunction suffers from or is infected by any bacterial or viral infaction (such as Rabies). In some embodiments, a subject in need of a method for reducing BBB permeability or dysfunction suffers from cerebral malaria.

In some embodiments, the present methods provide preventive measures for a subject susceptible of acquiring a brain disease or at risk of a brain disease deterioration. In some embodiments, a subject in need of preventive measures is in contact with patients afflicted with Meningitis. In some embodiments, a subject in need of preventive measures participates in athletic or sport activity that often results in brain injuries. In some embodiments, a subject in need of preventive measures suffers from epilepsy. In some embodiments, a subject in need of preventive measures suffers from multiple sclerosis. In some embodiments, a subject in need of preventive measures has high risk for being infected with HIV. In some embodiments, a subject in need of preventive measures is exposed to Rabies. In some embodiments, a subject in need of preventive measures is at risk of a brain injury. In some embodiments, a subject in need of preventive measures is at risk of a traumatic brain injury (TBI). In some embodiments, a subject in need of a method for enhancing and/or inducing BBB permeability suffers from De Vivo disease. In some embodiments, a subject to be treated with the compositions and methods as described herein is afflicted with high BBB permeability. In some embodiments, a subject to be treated with the compositions and methods as described herein is afflicted with hyper-permeability of the BBB. In some embodiments, a subject to be treated with the compositions/combinations and methods as described herein is in need of BBB closure and/or reduction of BBB permeability.

### Pharmaceutical Compositions

In some embodiments, the invention is directed to a composition comprising an N-Methyl D-Aspartate receptor (NMDA) receptor blocker and a Transforming Growth Factor beta (TGF-β) receptor antagonist. In some embodiments, the composition comprises an effective amount of an NMDA receptor blocker and an effective amount of a TGF-β receptor antagonist. In some embodiments, the composition comprises an effective amount of an NMDA receptor blocker and an effective amount of a TGF-β receptor antagonist and a carrier and/or diluent.

In some embodiments, the invention is directed to a pharmaceutical composition comprising as an active ingredient an effective amount of an NMDA receptor blocker and an effective amount of a TGF-β receptor antagonist, and a pharmaceutically acceptable carrier and/or diluent.

In some embodiments, the invention is directed to a pharmaceutical composition comprising as an active ingredient a therapeutically effective amount of Memantine or its analog and a therapeutically effective amount of Losartan or its analog, and a pharmaceutically acceptable carrier and/or diluent. In some embodiments, the memantine and losartan are formulated together within the pharmaceutical composition. In some embodiments, the memantine and losartan are formulated separately. As used herein, the term “analog” relates to a derivative or an isomer as described hereinabove.

For example, the term “pharmaceutically acceptable” can mean approved by a regulatory agency of the Federal or a state government or listed in the U.S. Pharmacopeia or other generally recognized pharmacopeia for use in animals, and more particularly in humans.

In some embodiments, the pharmaceutical composition described herein comprises the NMDA receptor blocker and the TGF-β receptor antagonist in the ratio of 1:1 to 1:15 (w/w). In some embodiments, the w/w ratio of the NMDA receptor blocker to the TGF-β receptor antagonist within the pharmaceutical composition is between 1:1 and 1:10, between 1:1 and 1:5, between 1:0.1 and 1:2between 1:0.5 and 1:2 between 1:0.1 and 1:5 between 1:0.1 and 1:10 between 1:0.1 and 1:15 between 1:0.1 and 1:20 including any range or value therebetween Each possibility represents a separate embodiment of the invention.

In some embodiments, the NMDA receptor blocker (such as memantine) is present at a concentration of at least 0.01 mg/ml, at least 0.1 mg/ml, at least 0.5 mg/ml, at least 1 mg/ml, at least 5 mg/ml, at least 10 mg/ml, at least 15 mg/ml, at least 20 mg/ml, at least 25 mg/ml, at least 30 mg/ml, at least 35 mg/ml, at least 40 mg/ml, at least 50 mg/ml, at least 60 mg/ml, at least 70 mg/ml, at least 80 mg/ml, at least 90 mg/ml, at least 100 mg/ml, or any range therebetween, within the pharmaceutical composition. In some embodiments, NMDA receptor blocker is present at a concentration of 0.1-1 mg/ml, 0.05-1.5 mg/ml, 1-5 mg/ml, 4-10 mg/ml, 6-12 mg/ml, 11-15 mg/ml, 12-20 mg/ml, 15-25 mg/ml, 20-35 mg/ml, 30-45 mg/ml, 40-60 mg/ml, 50-70 mg/ml, 60-80 mg/ml, 70-90 mg/ml, or 80-100 mg/ml or any range therebetween, within the pharmaceutical composition. Each possibility represents a separate embodiment of the invention.

In some embodiments, the TGF-β receptor antagonist (such as losartan) is present at a concentration of at least 0.01 mg/ml, at least 0.1 mg/ml, at least 0.5 mg/ml, at least 1 mg/ml, at least 5 mg/ml, at least 10 mg/ml, at least 15 mg/ml, at least 20 mg/ml, at least 25 mg/ml, at least 30 mg/ml, at least 35 mg/ml, at least 40 mg/ml, at least 50 mg/ml, at least 60 mg/ml, at least 70 mg/ml, at least 80 mg/ml, at least 90 mg/ml, at least 100 mg/ml, or any range there between, within the composition. In some embodiments, a TGF-β receptor antagonist is present at a concentration of 0.1-1 mg/ml, 0.05-1.5 mg/ml, 1-5 mg/ml, 4-10 mg/ml, 6-12 mg/ml, 11-15 mg/ml, 12-20 mg/ml, 15-25 mg/ml, 20-35 mg/ml, 30-45 mg/ml, 40-60 mg/ml, 50-70 mg/ml, 60-80 mg/ml, 70-90 mg/ml, or 80-100 mg/ml within the composition. Each possibility represents a separate embodiment of the invention.

In another aspect of the invention, there is a kit comprising a first pharmaceutical composition and a second pharmaceutical composition. In some embodiments, the first pharmaceutical composition comprises the NMDA receptor blocker and a pharmaceutically acceptable carrier and/or diluent. In some embodiments, the second pharmaceutical composition comprises the TGF-β receptor antagonist and a pharmaceutically acceptable carrier and/or diluent.

In some embodiments, the first pharmaceutical composition comprises memantine and a pharmaceutically acceptable carrier and/or diluent. In some embodiments, the second pharmaceutical composition comprises losartan and a pharmaceutically acceptable carrier and/or diluent.

In one embodiment, a composition or a pharmaceutical composition as described herein is/are topical composition/s. In one embodiment, a composition or or a pharmaceutical composition as described herein is/are oral composition/s. In one embodiment, a composition or or a pharmaceutical composition as described herein is/are injectable composition/s.

In some embodiments, a composition or a pharmaceutical composition is any of an emulsion, a liquid solution, a gel, a paste, a suspension, a dispersion, an ointment, a cream or a foam.

As used herein, the term “carrier” refers to a diluent, adjuvant, excipient, or vehicle with which the active ingredient is administered. Such carriers can be sterile liquids, such as water-based and oils, including those of petroleum, animal, vegetable or synthetic origin, such as peanut oil, soybean oil, mineral oil, sesame oil and the like, polyethylene glycols, glycerin, propylene glycol or other synthetic solvents.

Other non-limiting examples of carriers include, but are not limited to: terpenes derived from Cannabis, or total terpene extract from Cannabis plants, terpenes from coffee or cocoa, mint-extract, eucalyptus-extract, citrus-extract, tobacco-extract, anis-extract, any vegetable oil, peppermint oil, d-limonene, b-myrcene, a-pinene, linalool, anethole, a-bisabolol, camphor, b-caryophyllene and caryophyllene oxide, 1,8-cineole, citral, citronella, delta-3-carene, farnesol, geraniol, indomethacin, isopulegol, linalool, unalyl acetate, b-myrcene, myrcenol, 1-menthol, menthone, menthol and neomenthol, oridonin, a-pinene, diclofenac, nepafenac, bromfenac, phytol, terpineol, terpinen-4-ol, thymol, and thymoquinone. One skilled in the art will appreciate, that a particular carrier used within the pharmaceutical composition of the invention may vary depending on the route of administration.

In some embodiments, the carrier improves the stability of the active ingredient in a living organism. In some embodiments, the carrier improves the stability of the active ingredient within the pharmaceutical composition. In some embodiments, the carrier enhances the bioavailability of the active ingredient.

Water may be used as a carrier such as when the active ingredient is comprised by a pharmaceutical composition being administered intravenously. Saline solutions and aqueous dextrose and glycerol solutions can also be employed as liquid carriers, particularly for injectable solutions.

In some embodiments, the carrier is a liquid carrier. In some embodiments, the carrier is an aqeuous carrier.

Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; and agents for the adjustment of tonicity such as sodium chloride or dextrose are also envisioned. The carrier may comprise, in total, from 0.1% to 99.99999% by weight of the composition/s or the pharmaceutical composition/s presented herein.

In some embodiments, the pharmaceutical composition includes incorporation of any one of the active ingredients into or onto particulate preparations of polymeric compounds such as polylactic acid, polyglycolic acid, hydrogels, etc., or onto liposomes, microemulsions, micelles, unilamellar or multilamellar vesicles, erythrocyte ghosts, or spheroplasts. Such compositions may influence the physical state, solubility, stability, rate of in vivo release, and rate of in vivo clearance.

In some embodiments, the pharmaceutical composition is a liquid at a temperature between 15 to 45° C. In some embodiments, the pharmaceutical composition is a solid at a temperature between 15 to 45° C. In some embodiments, the pharmaceutical composition is a semi-liquid at a temperature between 15 to 45° C. It should be understood that the term “semi-liquid”, is intended to mean materials which are flowable under pressure and/or shear force. In some embodiments, semi-liquid compositions include creams, ointments, gel-like materials and other similar materials. In some embodiments, the pharmaceutical composition is a semi-liquid composition, characterized by a viscosity in a range from 31,000-800,000 cps.

Non-limiting examples of carriers for pharmaceutical compositions being in the form of a cream include but are not limited to: non-ionic surfactants (e.g., glyceryl monolinoleate glyceryl monooleate, glyceryl monostearate lanolin alcohols, lecithin mono- and di-glycerides poloxamer polyoxyethylene 50 stearate, and sorbitan trioleate stearic acid), anionic surfactants (e.g. pharmaceutically acceptable salts of fatty acids such as stearic, oleic, palmitic, and lauric acids), cationic surfactants (e.g. pharmaceutically acceptable quaternary ammonium salts such as benzalkonium chloride, benzethonium chloride, and cetylpyridinium chloride) or any combination thereof.

In some embodiments, the pharmaceutical composition being in the form of a cream further comprises a thickener.

Non-limiting examples of thickeners include, but are not limited to microcrystalline cellulose, a starch, a modified starch, gum tragacanth, gelatin, and a polymeric thickener (e.g. polyvinylpyrrolidone) or any combination thereof.

An embodiment of the invention relates to an NMDA receptor blocker and a TGF-β receptor antagonist, presented in unit dosage form and prepared by any of the methods well known in the art of pharmacy. In some embodiments, the unit dosage comprises a mixture of the NMDA receptor blocker (such as memantine) and the TGF-β receptor antagonist (such as losartan. In some embodiments, the NMDA receptor blocker is within a first unit dosage, and the TGF-β receptor antagonist is within a second unit dosage. In some embodiments, the unit dosage form is in the form of a tablet, capsule, lozenge, wafer, patch, ampoule, vial or pre-filled syringe.

In addition, in vitro assays may optionally be employed to help identify optimal dosage ranges. The precise dose to be employed in the formulation will also depend on the route of administration, and the nature of the disease or disorder, and should be decided according to the judgment of the practitioner and each patient's circumstances. Effective doses can be extrapolated from dose-response curves derived from in-vitro or in-vivo animal model test bioassays or systems. In some embodiments, the effective dose is determined as described hereinabove.

In one embodiment, the composition or the pharmaceutical composition of the present invention is administered in the form of a pharmaceutical composition comprising at least one of the active ingredients of this invention (e.g. an NMDA receptor blocker and a TGF-β receptor antagonist) together with a pharmaceutically acceptable carrier or diluent. In another embodiment, the composition of the invention can be administered either individually or together in any conventional oral, parenteral or transdermal dosage form.

As used herein, the terms “administering”, “administration”, and like terms refer to any method which, in sound medical practice, delivers a composition containing an active agent to a subject in such a manner as to provide a therapeutic effect.

In some embodiments, the pharmaceutical composition or the kit described herein (e.g., comprising an NMDA receptor blocker and a TGF-β receptor antagonist), is administered via oral (i.e., enteral), rectal, vaginal, topical, nasal, ophthalmic, transdermal, subcutaneous, intramuscular, intraperitoneal or intravenous routes of administration. The route of administration of the pharmaceutical composition or of the kit will depend on the disease or condition to be treated. Suitable routes of administration include, but are not limited to, parenteral injections, e.g., intradermal, intravenous, intramuscular, intralesional, subcutaneous, intrathecal, and any other mode of injection as known in the art. In addition, it may be desirable to introduce the pharmaceutical composition of the invention by any suitable route, including intraventricular and intrathecal injection; intraventricular injection may be facilitated by an intraventricular catheter, for example, attached to a reservoir. Pulmonary administration can also be employed, e.g., by use of an inhaler or nebulizer.

In some embodiments, the NMDA receptor blocker and/or the TGF-β receptor antagonist are independently mixed with a pharmaceutically acceptable carrier so that an effective dosage is delivered, based on the desired activity. The carrier can be in the form of, for example, and not by way of limitation, an ointment, cream, gel, paste, foam, aerosol, suppository, pad or gelled stick.

In some embodiments, for oral applications, the pharmaceutical composition or the kit is in the form of a tablets or a capsule, which can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate; or a glidant such as colloidal silicon dioxide. When the dosage unit form is a capsule, it can contain, in addition to materials of the above type, a liquid carrier such as fatty oil. In addition, dosage unit forms can contain various other materials which modify the physical form of the dosage unit, for example, coatings of sugar, shellac, or other enteric agents. In some embodiments, the tablet of the invention is further film coated. In some embodiment, oral application of the pharmaceutical composition or of the kit is in a form of drinkable liquid. In some embodiment, oral application of the pharmaceutical composition or of the kit is in a form of an edible product.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes.

In some embodiments, the composition, the pharmaceutical composition or the kit comprises incorporation of the active ingredient into or onto particulate preparations of polymeric compounds such as polylactic acid, polyglycolic acid, hydrogels, etc., or onto liposomes, microemulsions, micelles, unilamellar or multilamellar vesicles, erythrocyte ghosts, or spheroplasts. Such composition or pharmaceutical composition will influence the physical state, solubility, stability, rate of in vivo release, and rate of in vivo clearance.

In one embodiment, the present invention provides combined preparations. In one embodiment, “a combined preparation” defines especially a “kit” or a “kit of parts” in the sense that the combination partners as defined above can be dosed independently or by use of different fixed combinations with distinguished amounts of the combination partners i.e., simultaneously, concurrently, separately or sequentially. In some embodiments, the parts of the kit of parts can then, e.g., be administered simultaneously or chronologically staggered, that is at different time points and with equal or different time intervals for any part of the kit of parts. The ratio of the total amounts of the combination partners, in some embodiments, can be administered in the combined preparation. In one embodiment, the combined preparation can be varied, e.g., in order to cope with the needs of a patient subpopulation to be treated or the needs of the single patient which different needs can be due to a particular disease, severity of a disease, age, sex, or body weight as can be readily made by a person skilled in the art.

According to another embodiment, there is provided a combination of an NMDA receptor blocker and a TGF-β receptor antagonist, for use in the treatment of a disease or a disorder, wherein the NMDA receptor blocker and the TGF-β receptor antagonist are as described hereinabove. According to another embodiment, there is provided a combination of a pharmaceutical composition comprising an effective amount of the NMDA receptor blocker and a pharmaceutical composition comprising an effective amount of the TGF-β receptor antagonist, for use in the treatment of a disease or a disorder. In one embodiment, the combination of an NMDA receptor blocker and a TGF-β receptor antagonist is for use in the treatment of a disorder selected from the group consisting of: stroke, Alzheimer's disease, non-Alzheimer's neurodegenerative diseases, acute liver failure, multiple sclerosis, meningitis, HIV, diabetes, depressive and psychotic disorders, cerebral malaria, Parkinson's disease, traumatic and surgical brain injury, concussion, peripheral nerve injury, brain cancer, epilepsy and peripheral inflammatory pain.

According to another embodiment, there is provided a pharmaceutical composition comprising an effective amount of the TGF-β receptor antagonist for use in enhancing the therapeutic efficacy of the NMDA receptor blocker. According to another embodiment, there is provided a pharmaceutical composition comprising an effective amount of the TGF-β receptor antagonist for use in treatment of a disease in a subject amenable for treatment by the NMDA receptor blocker.

According to some embodiments, the NMDA receptor blocker and the TGF-β receptor antagonist are administered concurrently. In some embodiments, the NMDA receptor blocker and the TGF-β receptor antagonist are administered sequentially. In some embodiments, the NMDA receptor blocker and the TGF-β receptor antagonist are administered subsequently.

In one embodiment, it will be appreciated that the NMDA receptor blocker and the TGF-β receptor antagonist of the present invention can be provided to the individual with additional active agents to achieve an improved therapeutic effect as compared to treatment with each agent by itself. In some embodiments, an additional active agent is any therapeutically active ingredient with the proviso that it is not an anti-epileptic compound (e.g. valproate).

In another embodiment, measures (e.g., dosing and selection of the complementary agent) are taken to adverse side effects which are associated with combination therapies.

In one embodiment, depending on the severity and responsiveness of the condition to be treated, dosing can be of a single or a plurality of administrations, with course of treatment lasting from several days to several weeks or until cure is affected or diminution of the disease state is achieved.

In some embodiments, the composition of the present invention is administered in a therapeutically safe and effective amount. As used herein, the term “safe and effective amount” refers to the quantity of a component which is sufficient to yield a desired therapeutic response without undue adverse side effects, including but not limited to toxicity, such as calcemic toxicity, irritation, or allergic response, commensurate with a reasonable benefit/risk ratio when used in the presently described manner. In another embodiment, a therapeutically effective amount of an NMDA receptor blocker and a TGF-β receptor antagonist is the amount of the mentioned herein NMDA receptor blocker and a TGF-β receptor antagonist necessary for the in vivo measurable expected biological effect. The actual amount administered, and the rate and time-course of administration, will depend on the nature and severity of the condition being treated. Prescription of treatment, e.g. decisions on dosage, timing, etc., is within the responsibility of general practitioners or specialists, and typically takes account of the disorder to be treated, the condition of the individual patient, the site of delivery, the method of administration and other factors known to practitioners. Examples of techniques and protocols can be found in Remington: The Science and Practice of Pharmacy, 21st Ed., Lippincott Williams & Wilkins, Philadelphia, Pa., (2005). In some embodiments, preparation of effective amount or dose can be estimated initially from in vitro assays. In one embodiment, a dose can be formulated in animal models and such information can be used to more accurately determine useful doses in humans.

In one embodiment, toxicity and therapeutic efficacy of the active ingredients described herein can be determined by standard pharmaceutical procedures in vitro, in cell cultures or experimental animals. In one embodiment, the data obtained from these in vitro and cell culture assays and animal studies can be used in formulating a range of dosage for use in human. In one embodiment, the dose used in the in vitro-experiment (also referred to as an animal equivalent dose) can be converted into a human dose, such as by using a conversion factor. Various conversion factors are known in the art. [See e.g., Rockville, Md.: Guidance for Industry: Estimating the Maximum Safe Starting Dose in Adult Healthy Volunteer, US Food and Drug Administration; 2005]. In one embodiment, the human dose is calculated by dividing the rat equivalent dose by 6.2 (conversion factor). In one embodiment, the dosages vary depending upon the dosage form employed and the route of administration utilized. In one embodiment, the exact formulation, route of administration and dosage can be chosen by the individual physician in view of the patient's condition. [See e.g., Goodman and Gilman's The Pharmacological Basis of Therapeutics, 13th Ed., McGraw-Hill/Education, New York, N.Y. (2017)].

Pharmaceutical compositions containing the presently described NMDA receptor blocker and the TGF-β receptor antagonist as the active ingredient can be prepared according to conventional pharmaceutical compounding techniques. See, for example, Remington: The Science and Practice of Pharmacy, 22nd Ed., Pharmaceutical Press, Philadelphia, Pa. (2012).

In one embodiment, compositions including the preparation of the present invention formulated in a compatible pharmaceutical carrier are prepared, placed in an appropriate container, and labeled for treatment of an indicated condition.

In one embodiment, compositions of the present invention are presented in a pack or dispenser device, such as an FDA approved kit, which contains, one or more unit dosages forms containing the active ingredient. In one embodiment, the pack, for example, comprises metal or plastic foil, such as a blister pack. In one embodiment, the pack or dispenser device is accompanied by instructions for administration. In one embodiment, the pack or dispenser is accommodated by a notice associated with the container in a form prescribed by a governmental agency regulating the manufacture, use or sale of pharmaceuticals, which notice is reflective of approval by the agency of the form of the compositions or human or veterinary administration. Such notice, in one embodiment, is labeling approved by the U.S. Food and Drug Administration for prescription drugs or of an approved product insert.

Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a”, “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise”, “include” and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

### EXAMPLES

Generally, the nomenclature used herein, and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds.) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document

[symbol]

### Materials and Methods

### Chemicals and Antibodies

All experimental procedures in animals were approved by the Ben-Gurion University ethics committee for animal testing. Unless otherwise mentioned, all materials were purchased from Sigma-Aldrich ltd. (Rehovot, Israel). Surgical procedures in male Sprague-Dawley rats (200-380 gr body weight) were performed as previously reported (Prager et al., 2010). Rats were deeply anesthetized by intraperitoneal administration of ketamine (100 mg/ml, 0.08 ml/100 gr) and xylasine (20 mg/ml, 0.06 ml/100 gr) or isoflurane inhalation (1-2% in O₂). The tail vein was catheterized, and animals were placed in a stereotactic frame under a SteREO Lumar V12 fluorescence microscope (Zeiss Ltd., Oberkochen, Germany). Body temperature was continuously monitored and kept stable at 37±0.5° C. using a feedback-controlled heating pad (Physitemp Ltd., Clifton, N.J., USA). Heart rate, breath rate and oxygen saturation levels were continuously monitored using MouseOx (STARR Life Sciences Ltd. Oakmont, Pa., USA). A cranial section (4 mm caudal, 2 mm frontal, 5 mm lateral to bregma) was removed over the right sensory-motor cortex. The dura and arachnoid layers were removed, and the exposed cortex was continuously perfused with artificial cerebrospinal fluid (ACSF) containing (in mM): 124 NaCl, 26 NaHCO₃, 1.25 NaH₂PO₄, 2 MgSO₄, 2 CaCl₂), 3 KCl, and 10 Glucose (pH 7.4). For neuronal activity blocking, tetrodotoxin (TTX, 10 μM), 6-Cyano-2,3-dihydroxy-7-nitro-quinoxaline (CNQX, 50 μM)(Yoshiyama, Roppolo, & WC, 1995), D-(−)-2-amino-5-phosphonopentanoic acid (AP5, 100 μM) were added to the ACSF. To induce prolonged seizures (status epilepticus (SE)), 4AP (500 μM) or picrotoxin (PTX, 100 μM) were added to the ACSF. To block transforming growth factor beta (TGF-β) receptors, 2-(3-(6-Methylpyridin-2-yl)-1H-pyrazol-4-yl)-1,5-naphthyridine was added to ACSF (SJN2511, SJN, 0.3 mM, Seleckchem ltd., Houston, Tex., USA). In several experiments, bovine serum albumin (BSA, 0.2 mM), angiotensin II (ATII, 70 μM, Adoq ltd., Irvine, Calif., USA) or TGF-β1 (10 ng/ml, Peprotech ltd., Rehovot, Israel) were added to the ACSF. As BBB protective therapy, the N-methyl-D-aspartate (NMDA) receptor (NMDA-R) antagonist (NMDAr-A) 3,5-dimethyladamantan-1-amine (Memantine, 25 mg/ml in 0.9% NaCl, 40 mg/kg) and/or the TGF-β/angiotensin receptor type 1 antagonist (2-Butyl-4-chlor-1-{[2′-(1H-tetrazol-5-yl)-4-biphenylyl]methyl}-1H-imidazol-5-yl) methanol (Losartan (Los), 25 mg/ml in 0.9% NaCl, 60 mg/kg), were administered either by i.p injection or by loading and implantation of osmotic pumps (Alzet Ltd., Cupertino, Calif. USA), or by supplemention to drinking water (40 mg/kg and 2 g/l respectively) or by both. Electrocorticography (ECoG) was recorded using a telemetric system (Data Sciences International Ltd., St. Paul, Minn. USA). A bi-polar transmitter was implanted with one electrode attached to an intra-cranial screw adjacent to the exposed cortex, and the second placed over the exposed cortex while secured with bone wax (Ethicon ltd., Somerville, N.J., USA) and dental cement (GC America ltd., Alsip, Ill., USA).

### Fluorescent Angiography and Quantification of Vascular Permeability

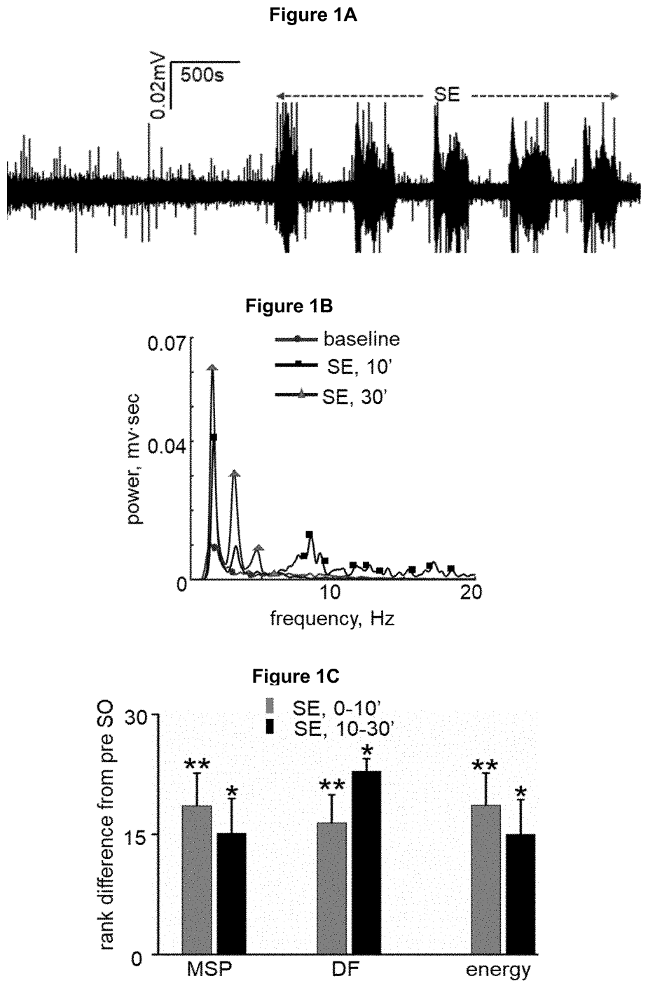

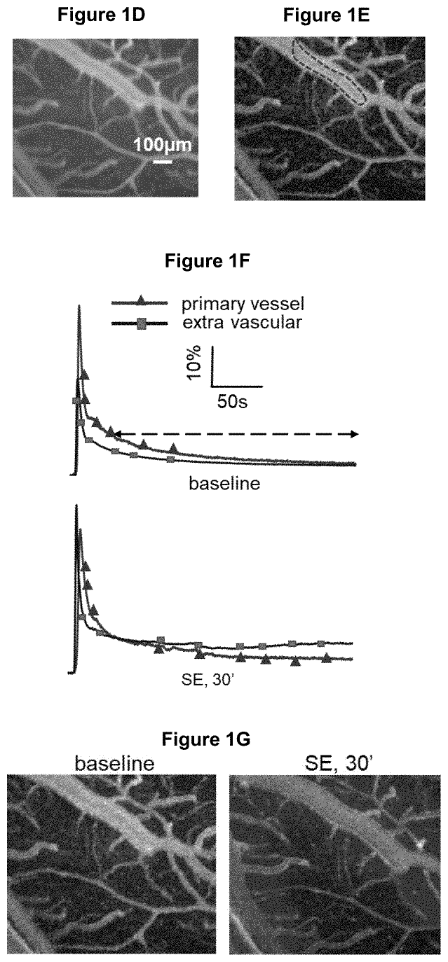

Dynamic imaging of regional cerebral blood flow (rCBF) and BBB permeability measurements were performed based on previous reports (Prager et al., 2010) with additions to the image analysis methods. The BBB impermeable tracer, sodium fluorescein (NaFlu), was administered i.v, while full-resolution images of cortical surface vessels were acquired (with excitation at 470±35 nm, at 5 frames/sec, 658×496 or 512×512 pixel, using EMCCD camera, Andor Technology, DL-658 M-TIL, Belfast, UK, FIG. 1D) before, during and after injection of the tracer. Offline image analysis was carried out using in-house developed MATLAB (MathWorks, Natick, Mass., USA) algorithms, and included: resizing (128×128 pixel), image registration (Guizar-Sicairos et al., 2008), and threshold segmentation using noise filtration, hole-filling and adaptive threshold to produce a binary image, separating blood vessels from extra-vascular (EV) regions (FIG. 1E). A primary vessel was then manually selected (FIG. 1E). Signal intensity changes over time and space were analyzed so that each pixel was represented by intensity vs time (IT) curve. A compartmental IT curve was created by spatially averaging IT values in a chosen compartment (FIG. 1F). An EV/pixel BBB permeability index (PI) was calculated as the ratio between EV/pixel IT curve, and the vascular IT curve (vascular input function-VIF), from the point of the second decline phase to the end of the measurement (˜250-300 sec, FIG. 1F):

$$PI = \frac{1}{T} {\int }_{{t}_{cr}}^{{t}_{end}} \frac{{I}_{compartment}}{{I}_{VIF}} ( t ) dt T = {t}_{end} - {t}_{cr} .$$

The PI indicates the tracer's transfer level from the vessel to a chosen pixel or to the EV space. Specifically, EV PI>1 indicates BBB dysfunction. This method was validated in well-established models of BBB dysfunction such as cortical perfusion of sodium deoxycholate, photo-induced stroke (Prager et al., 2010) and SE (FIG. 1F-G).

### Electrocorticography Recording and Analysis

In-house MATLAB scripts were used to display and record signals (FIG. 1A), as well as for post-processing. The signal x(t) was sampled at 200 Hz and filtered using a simulated Butterworth filter, so to display only the 1-40 Hz frequency band. Mean spectral power (MSP) was calculated as

$$\hat{S} = \frac{1}{Δ f} {\int }_{Δ f} S ( f ) ,$$

where

$$S ( f ) = {\int }_{- ∞}^{∞} E [ x ( t ) , x ( t + τ ) ] {e}^{- j 2 π f τ} d τ$$

(FIG. 1B) and E[x(t)] is the signal expectancy. The dominant frequency (DF) was calculated as the frequency corresponding to the maximum value of S(f). Signal energy was calculated as

$${\int }_{- ∞}^{∞} {\vert x ( t ) \vert}^{2} dt .$$

Baseline recording was performed prior to 4AP/PTX application, from which a period (17.78±1.34 min, mean±squared error of the mean) was selected as representing quiescence. Shifts in MSP, energy and DF, were then calculated relative to quiescence, for 1 min intervals. Statistical analysis was applied to shifts in ECoG features (Delorme et al., 2007; Tadel et al., 2011) as values were ranked according to the Mann-Whitney U ranking system (Nachar, 2008). The differences in mean rank between pre and post seizure onset, were calculated for all three parameters (FIG. 1C). Additionally, an in-house developed, MATLAB simulated, seizure detector and counter was employed, based on positive rank differences in at least two of the three parameters.

### Halogen-Induced Transcranial Photothrombosis

Rats were deeply anesthetized by intraperitoneal administration of ketamine (100 mg/ml, 0.08 ml/100 gr) and xylasine (20 mg/ml, 0.06 ml/100 gr) and placed in a stereotactic frame. The tail vein was catheterized, and a scalp incision was made. Animals were i.v. injected with the photo-reactive substance, 4,5,6,7-tetrachloro-2′,4′,5′,7′-tetraiodofluorescein (Rose Bengal-RB, 7.5 mg/ml in 0.9% NaCl, 0.133 ml/100 g), and subjected to photothrombosis (PT) via halogen illumination on an intact cranium.

### Brain MRI for Evaluation of Infarct and Vascular Permeability

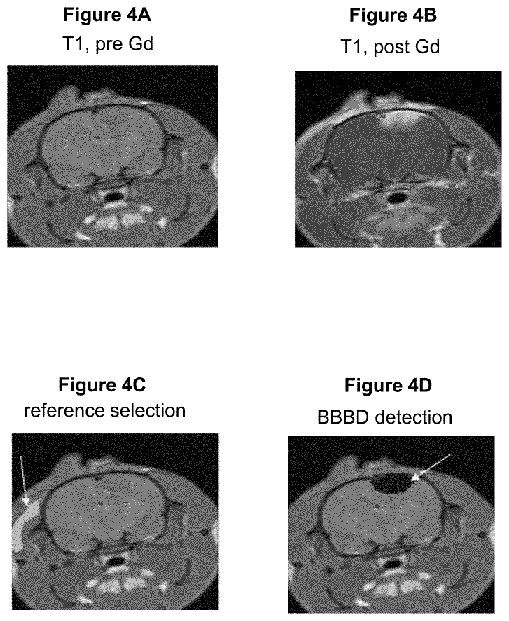

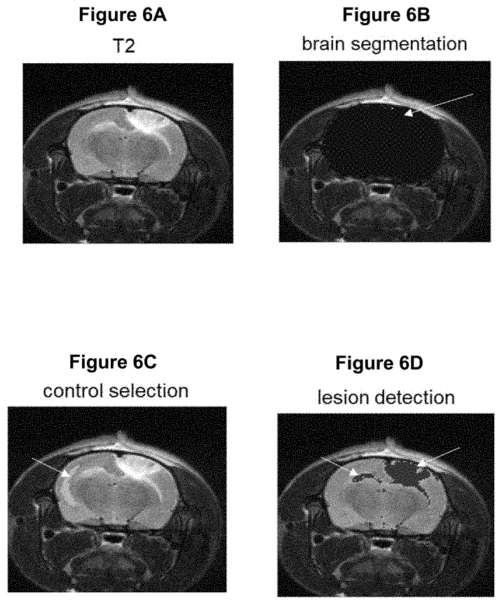

Naïve animals were randomly grouped according to treatment selection: vehicle-animals treated with 0.9% NaCl, memantine-animals treated with 40 mg/kg of memantine stand-alone, Los-animals treated with 60 mg/kg of Los stand alone, memantine+Los-animals treated with the combined treatment (40 mg/kg and 60 mg/kg of memantine and Los respectively). Animals were anesthetized by intraperitoneal administration of ketamine (100 mg/ml, 0.08 ml/100 gr) and xylasine (20 mg/ml, 0.06 ml/100 gr) and were subjected to halogen-induced photothrombosis, as described above. Immediately following infarct induction, treatment was administered according to group assignment by way of osmotic pump implantation to the peritoneal cavity and drinking water supplementation (see Animal handling). 48 h following infarct induction, animals were anesthetized via isoflurane inhalation (1-2% in 02) and placed in a μ-MRI scanner (0.7T, Aspect Imaging ltd., Shoham Israel). Fast spin-echo T2 and spin-echo T1-weighted imaging sessions were performed and followed by i.m. administration of the gadolinium-based, albumin-binding contrast agent gadofosveset (Gd, ABLAVAR, Lantheus ltd., MA USA). Contrast-enhanced T1-weighted imaging session was performed following 30 min. Offline, analyses of contrast enhanced T1-weighted imaging (FIG. 4), and T2-weighted imaging (FIG. 6) were performed following image segmentation (FIG. 6B) calculated from T2-weighted imaging by identifying voxels within brain signal histogram and out of peripheral signal histogram. Total volumes of brain lesion and abnormally high-level permeability (BBB dysfunction) were detected offline using in-house developed MATLAB algorithms. In contrast enhanced T1-weighted imaging, the environmental signal histogram was calculated and compared to the signal histogram of a reference region acquired from temporal muscle (a non-BBB containing tissue, FIG. 4C), and the parallel environmental signal from the prior, non-contrast enhanced imaging session. BBB dysfunction voxels were identified as having a higher (according to mean Mann-Whitney U rank) self and environmental contrast enhanced signal than the non-contrast enhanced parallel signal, and a higher environmental contrast-enhanced signal than the reference signal. The relative BBB dysfunction volume was calculated as the ratio between the number of BBB dysfunction voxels and number of whole brain voxels.

In T2-weighted imaging, the signal histogram in a 3×3 environment around each voxel (environmental signal) was calculated and compared to the signal histogram of a reference control region acquired from the hemisphere contralateral to the infarcted hemisphere (FIG. 6C). Lesion voxels were identified (FIG. 6D) as having a higher (according to Mann-Whitney U rank) self and environmental signal than the control signal. The relative lesion volume was calculated as the ratio between the number of lesion voxels and number of whole brain voxels.

### Experimental Design

Naïve animals were randomly selected for treatment. Data were analyzed blindly and identically (regardless of treatment selection) using MATLAB algorithms developed in-house and validated in advance.

### Statistical Analysis

Unless otherwise mentioned, numerical data are expressed as mean±squared error of the mean (SEM). All comparisons were made using two-tailed Mann-Whitney-U test (Mann-Whitney). P=0.05 was defined as the level of significance. Statistical analysis was performed using SPSS (IBM, Armonk, N.Y., USA).

### Example 1

### NMDA Antagonists Prevent Fast BBB Dysfunction Following 4AP-Induced Seizures

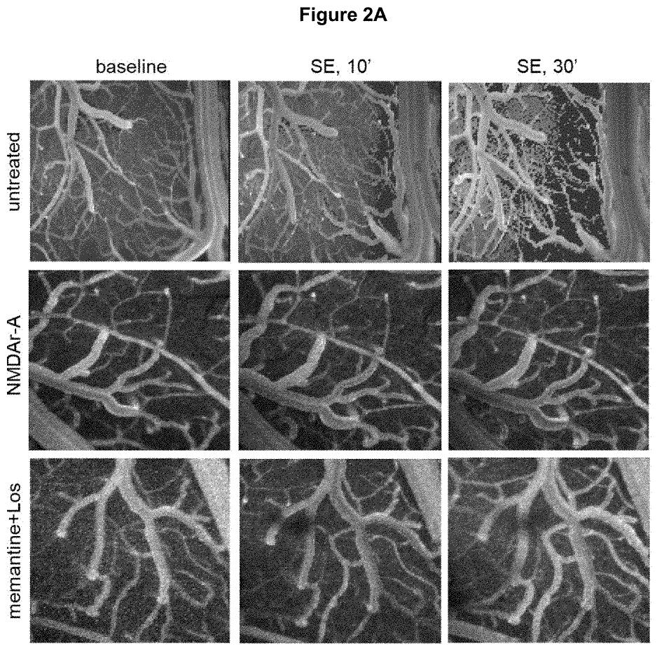

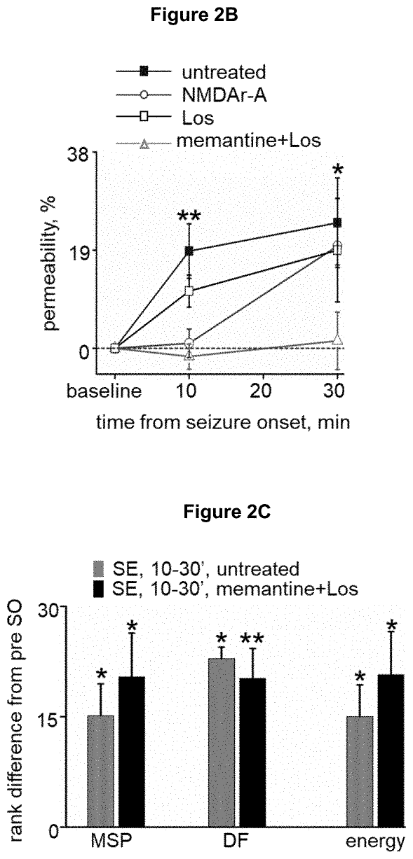

In this experiment, the inventors tested the effect of NMDA-R antagonists on BBB dysfunction induced by recurrent seizures. Recurrent Seizures were induced by 4AP or by PTX (FIG. 1A). Electrocorticographic recording (ECoG) during seizure activity revealed, as expected, increased mean spectral power (MSP), dominant frequency (DF) and energy compared with analysis of baseline recording (prior to the initiation of seizures) with the same duration (18.57±4.06%, 16.45±3.51% and 18.64±4.03% for 10 min from seizure onset, and 15.13±4.34%, 22.9±1.55% and 15.01±4.32% for 30 min, P=0.002 and P=0.01 respectively, (FIGS. 1B-C). The BBB impermeable tracer, sodium fluorescein was intravenously administered, while full resolution images of the exposed rat cortex were acquired (FIG. 1D). Offline image analysis was used to produce a binary image delineating blood vessels from extravascular regions, thus facilitating selection of a primary vessel for further analysis (FIG. 1E). The fluorescent signal intensity of this vessel over time and the signal intensity of the surrounding extravascular region over time were determined to calculate the BBB permeability index (PI, FIG. 1F). Extravascular permeability increase above baseline could be detected at 30 minutes from seizure onset (FIG. 1G), as well as before (18.82±5.28%, P<0.001 and 24.3±8.66%, P=0.001 for 10 and 30 min, respectively, n=12, FIG. 2A-B). The period from 4AP/PTX application to the onset of seizures, was not accompanied by a shift from baseline in the above mentioned ECoG parameters (−9.75±5.63% for MSP, 1.22±4.2% for DF and −10.29±5.82% for energy), nor was a change in PI observed (−0.89±2.61%).

To test the role of glutamate in seizure-induced BBB dysfunction, in one set of experiments the selective NMDA-R antagonist (NMDAr-A), AP5 was added to the perfusion solution (n=5), and in another set of experiments the clinically approved memantine was administered (i.p, n=5) at two time points for each animal: immediately following 4AP application and after seizure onset. The leak of peripherally-injected fluorescein (representing BBB dysfunction), measured at 10 min from seizures onset, was prevented in AP5 treated animals (−1.19±4.71% shift in PI from baseline, P=0.02 in comparison to untreated animals) and in memantine-treated animals (3.14±3.1%, P=0.03 in comparison to untreated; FIG. 2A-B). When angiography was repeated at 30 min following seizure onset, vascular permeability was increased similarly to the untreated group, in both AP5 and memantine-treated animals (18.78±6.93% and 20.86±3.78, n=4 and n=4, P=0.89 and P=0.48 compared to untreated, respectively FIG. 2A-B). No differences in permeability were found between the locally perfused AP5 and peripherally-injected memantine-treated animals at either 10 min (P=0.6) or 30 min (P=0.77) from seizure onset (data not shown). These findings suggest that while NMDA-R activation governs the immediate phase of seizure-induced BBB dysfunction, an independent, non-NMDA-R mediated mechanism underlies a slower increase in permeability.

### Example 2

### Seizure-Induced Slow BBB Dysfunction is Mediated by TGF-β Signalling

In search for an additional molecular pathway, whose seizure-induced activation could lead to vascular leakage, the inventors initially focused on angiotensin receptor type 1 activation in the endothelial membrane. The renin-angiotensin hormonal system regulates blood pressure and body fluid homeostasis. Angiotensinogen is cleaved by renin to angiotensin I, which is then converted to angiotensin II (ATII) by angiotensin-converting enzyme. It is known in the art, that ATII is upregulated during repeated seizures in experimental animals as well as in humans. To test this, 4AP was perfused to the exposed cortex, while i.p. administering both memantine and losartan (Los)—a clinically approved, angiotensin receptor type 1 antagonist, to achieve both fast and slow-phase BBB protection. Under these conditions, no increase in permeability was measured during 10- and 30 min follow-up after seizure onset (−1.6±2.45% and 1.45±5.57%, P=0.002 and P=0.02 in comparison to untreated group respectively, n=9, FIG. 2A-B). Treatment with Los alone did not prevent both fast and slow seizure-induced BBB dysfunction (11.08±3.1% and 19±10.01% shift in PI respectively, n=5, P=0.67 and P=0.71 in comparison to untreated group respectively (FIG. 2B).

To test whether BBB permeability was prevented because seizure activity was suppressed, the inventors evaluated the shifts from baseline in mean spectral power, dominant frequency and signal energy following the onset of seizures. Recurrent seizures were associated with a significant increase in all 3 ECoG parameters. Both combined and stand-alone treatment were accompanied by recurrent seizures (data not shown). Neither stand-alone (data not shown) nor the combined treatment resulted in a diminished impact for all 3 ECoG parameters (FIG. 2C), suggesting no blocking of seizure activity as well as a direct vascular protecting effect.

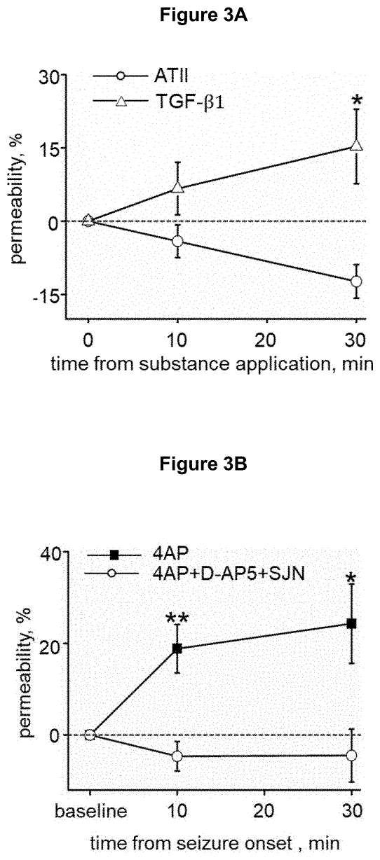

To test the relative BBB dysfunction-inducing role of ATII, TGF-β1, and serum albumin the inventors added the specific agonists to the ACSF perfusing the rat cortex, while blocking neuronal activity and synaptic transmission. ECoG recordings and analysis confirmed the absence of seizures or epileptiform activity under these conditions (data not shown). Vascular permeability to NaFlu was measured at 10 min (fast-phase) and 30 min (slow-phase) after adding the agonists. Results were compared to the shift in permeability observed with perfusion of ACSF alone for 60-120 min (−5.11±3.07, n=16, P=0.52 in comparison to baseline, Mann-Whitney). No change in permeability levels was observed when ATII was added (−4.11±3.35%, P=0.78 in comparison to ACSF perfusion and −12.33±3.45%, P=0.3, for 10 min and 30 min respectively, n=4, Mann-Whitney, FIG. 3A), indicating that ATII activation is not likely involved in acute-phase seizure-induced BBB dysfunction. Additionally, incubation with bovine-serum albumin failed to raise vascular permeability to NaFlu following application (−2.55±4.6% and −0.33±8.36%, n=4, P=0.64 respectively, Mann-Whitney). When TGF-β1 was added to the perfusion solution, vascular permeability gradually increased and became significant at 30 min from addition of the agonist (15.3±7.62%, n=7, P=0.02 compared to ACSF perfusion, and P=0.03 in comparison to ATII incubation, Mann-Whitney, FIG. 3A). In other experiments, 4AP was perfused to the exposed cortex, while i.p. administering both AP5 and SJN, a selective TGF-β receptor antagonist.

The joint administration (e.g., a combination) of both AP5 and SJN resulted in the prevention of both fast- and slow-phase BBB opening to NaFlu (At 10 min: −4.63±3.21% shift in PI vs. 18.82±5.28% for untreated animals, P=0.001 in comparison to untreated group; At 30 min: −4.47±5.76% vs. 24.3±8.66%, P=0.01; n=7, FIG. 3B).

### Example 3

### Memantine and Los Combined Treatment Diminishes Sub-Acute Phase Stroke-Induced BBB Dysfunction and Brain Oedema

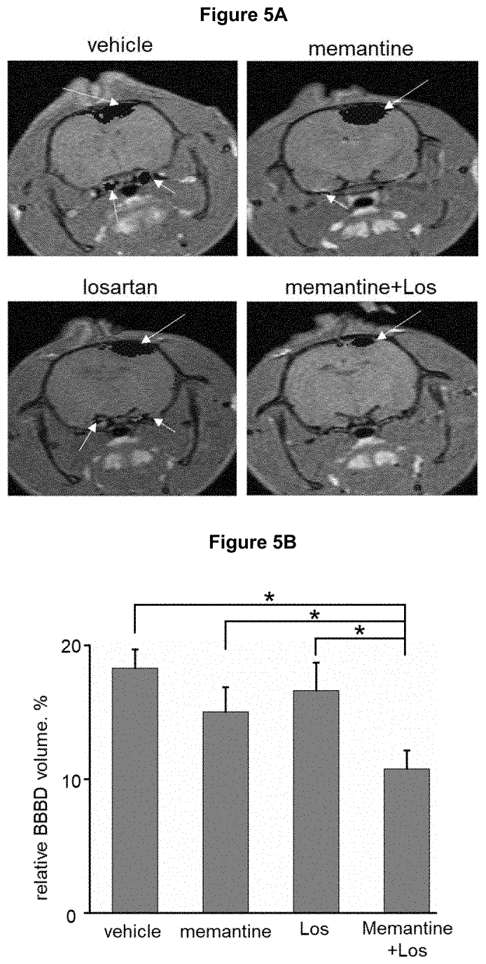

Further to the effectiveness of memantine and Los to prevent acute-phase seizure-induced BBB dysfunction, the inventors examined whether this combination, given at acute-phase, could also mitigate sub-acute phase BBB dysfunction and related brain edema following infarct induction. Animals were subjected to photothrombosis (PT) via halogen illumination on an intact cranium. Immediately following infarct induction, animals were implanted with osmotic pumps (see Materials and Methods) containing vehicle (0.9% NaCl, n=5), memantine stand-alone (memantine, 40 mg/kg n=8), losartan stand-alone (losartan, 60 mg/kg, n=5) and memantine and losartan combination (memantine+Los, n=6). T1-weighted MRI of the rat head 48 h following infarct induction was performed before (FIG. 4A), and after (FIG. 4B) administration of gadofosveset and a manual selection of a reference region in the temporal muscle (FIG. 4C), as well as detection of brain voxels with high-level permeability in comparison to reference (FIG. 4D), were made. Here, the combined treatment resulted in diminished relative BBB dysfunction volume compared to vehicle and both memantine and losartan stand-alone treated animals (10.76±1.38%, n=6, vs. 18.3±1.4%, n=5, 15.03±1.83%, n=7, and 16.63±2.09%, n=6, P=0.01, P=0.046 and P=0.04 respectively, FIG. 5A-B). These results indicate that BBB protection, provided by the combined treatment at acute-phase pathology, continues and alleviates sub-acute phase implications associated with BBB dysfunction in brain disease, while each stand-alone treatment fails to do so.

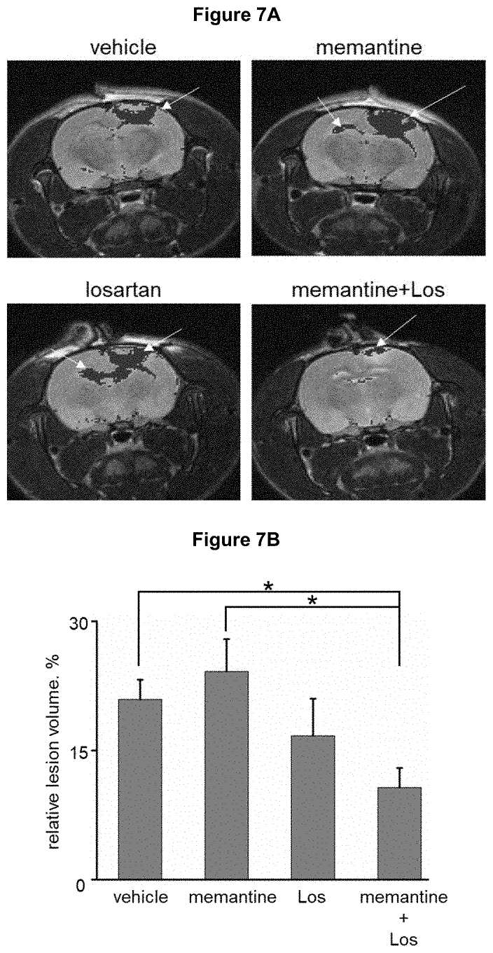

The inventors also performed T2-weighted MRI of the rat head, 48 h following infarct induction (FIGS. 6A-B), and a manual selection of a control region (FIG. 6C), as well as a detection of over-enhanced brain voxels in comparison to control (FIG. 6D), were made. Combined memantine-losartan treatment resulted in decreased relative lesion volume (FIG. 7A), in comparison to vehicle and memantine stand-alone but not losartan stand-alone treated animals (10.68±2.29%, n=6 vs. 20.94±2.28%, n=5, 24.18±3.75%, n=8, and 16.7±4.32%, n=8, P=0.01, P=0.04 and P=0.302 respectively, FIG. 7A-B).

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

1. A pharmaceutical composition comprising a therapeutically effective amount of an N-Methyl D-Aspartate receptor (NMDA) receptor blocker and a therapeutically effective amount of a Transforming Growth Factor beta (TGF-β) receptor antagonist.

2. The pharmaceutical composition of claim 1, wherein said NMDA receptor blocker is selected from the group consisting of: Memantine, D-2-amino-5-phosphonopentanoate (AP5), 2-amino-7-phosphonoheptanoic acid (AP7), 3-[(R)-2-carboxypiperazin-4-yl]-prop-2-enyl-1phosphonic acid, (2S,4R)-4-(phosphonomethyl)piperidine-2-carboxylic acid, Amantadine, Nitromemantine, and Symmetrel including any derivative, isomer or a combination thereof.

3. The pharmaceutical composition of claim 1, wherein said TGF-β receptor antagonist is selected from the group consisting of: Losartan, 4-(5-Benzol[1,3]dioxol-5-yl-4-pyridin-2-yl-1H-imidazol-2-yl)-benzamide, 2-(3-(6-Methylpyridin-2-yl)-1H-pyrazol-4-yl)-1,5-naphthyridine, Candesartan, and Telmisartan including any derivative, isomer or a combination thereof.

4. The pharmaceutical composition of claim 1, wherein said NMDA receptor blocker and said TGF-β receptor antagonist are present in said composition at a ratio ranging from 1:0.1 to 1:15.

5. The pharmaceutical composition claim 1, further comprising a pharmaceutically acceptable carrier.

6. (canceled)

7. (canceled)

8. (canceled)

9. (canceled)

10. A method for reducing the BBB permeability in a subject in need thereof, comprising contacting the subject with an effective amount of an NMDA receptor blocker and an effective amount of a TGF-β receptor antagonist thereby reducing the BBB permeability in the subject.

11. The method of claim 10, wherein said NMDA receptor blocker and said TGF-β receptor antagonist are administered at a ratio ranging from 1:0.1 to 1:15.

12. A method for increasing or prolonging the therapeutic efficacy of an NMDA receptor blocker in a subject treated with an NMDA receptor blocker, comprising administering to said subject a pharmaceutical composition comprising a TGF-β receptor antagonist.

13. The method of claim 10, wherein said NMDA receptor blocker is administered at a dosage of 0.1-40 mg/kg.

14. The method of claim 10, wherein said TGF-β receptor antagonist is administered at a dosage of 0.1-60 mg/kg.

15. The method of claim 10, wherein said NMDA receptor blocker is selected from the group consisting of: Memantine, D-2-amino-5-phosphonopentanoate (AP5), 2-amino-7-phosphonoheptanoic acid (AP7), 3-[(R)-2-carboxypiperazin-4-yl]-prop-2-enyl-1phosphonic acid, (2S,4R)-4-(phosphonomethyl)piperidine-2-carboxylic acid, Amantadine, Nitromemantine, and Symmetrel including any derivative, isomer or a combination thereof.

16. The method of claim 10, wherein said TGF-β receptor antagonist is selected from the group consisting of: Losartan, 4-(5-Benzol[1,3]dioxol-5-yl-4-pyridin-2-yl-1H-imidazol-2-yl)-benzamide, 2-(3-(6-Methylpyridin-2-yl)-1H-pyrazol-4-yl)-1,5-naphthyridine, Candesartan, and Telmisartan including any derivative, isomer or a combination thereof.

17. The method of claim 10, wherein said subject is afflicted with BBB dysfunction.

18. The method of claim 17, wherein said BBB dysfunction is selected from the group consisting of: epilepsy, traumatic brain injury, neurodegenerative diseases and brain ischemia.

19. The method of claim 12, wherein said TGF-β receptor antagonist is administered at a dosage of 0.1-60 mg/kg.

20. The method of claim 12, wherein said TGF-β receptor antagonist is selected from the group consisting of: Losartan, 4-(5-Benzol[1,3]dioxol-5-yl-4-pyridin-2-yl-1H-imidazol-2-yl)-benzamide, 2-(3-(6-Methylpyridin-2-yl)-1H-pyrazol-4-yl)-1,5-naphthyridine, Candesartan, and Telmisartan including any derivative, isomer or a combination thereof.

21. The method of claim 12, wherein said subject is afflicted with BBB dysfunction.

22. The method of claim 17, wherein said BBB dysfunction is selected from the group consisting of: epilepsy, traumatic brain injury, neurodegenerative diseases and brain ischemia.
